(19) United States
(12) Patent Application Publication
(10) Pub. No.: US 2024/0191265 A1
(43) Pub. Date: Jun. 13, 2024

(54) Beta-Lactone Production Through Bioconversion and Methods Related Thereto

(71) Applicant: ExxonMobil Technology and Engineering Company, Baytown, TX (US)

(72) Inventors: Xiaozhou Zhang, Bridgewater, NJ (US); Glen E. Alliger, Houston, TX (US); Yuchen Liu, Annandale, NJ (US)

(21) Appl. No.: 18/509,425

(22) Filed: Nov. 15, 2023

Related U.S. Application Data
(60) Provisional application No. 63/385,234, filed on Nov. 29, 2022.

(51) Int. Cl. C12P 7/62

(52) U.S. Cl. CPC C12P 7/62

(57) ABSTRACT

Methods of β-lactone production, including the integrated bioconversion of β-lactone production and methods related thereto. Methods include reacting a feedstock and a recombinant microorganism harboring one or more genes encoding enzymes involved in the metabolic pathway of a 3-hydroxycarboxylic acid, thereby producing a 3-hydroxycarboxylic acid having C₁-C₁₀ alkyl groups. Thereafter, reacting the 3-hydroxycarboxilic acid with a bioengineered natural β-lactone synthetase, the bioengineered β-lactone synthetase engineered to comprise a substrate binding pocket capable of accepting the 3-hydroxycarboxylic acid having C₁-C₁₀ alkyl groups, thereby producing a β-lactone having C₁-C₁₀ alkyl groups.

### CROSS REFERENCE TO RELATED APPLICATIONS

This application claims the benefit of and priority to U.S. Provisional Application No. 63/385234 filed Nov. 29, 2022, the disclosure of which is incorporated herein by reference.

### FIELD OF INVENTION

This application relates to beta-lactone production and, more particularly, to integrated bioconversion of beta-lactone production and methods related thereto.

### BACKGROUND

Beta-lactones (β-lactones) are four-membered heterocycles with high ring-strain. They act as reactive electrophilic biologically active scaffolds and serve as versatile synthetic intermediates, with many having potent bioactivity against bacteria, fungi, and/or human cancer cell lines.

However, it can be a challenge to synthesize β-lactones through traditional chemical catalysis, due to their particular chemical structure (e.g., 4 or greater ring structures). Naturally occurring β-lactone synthetases facilitate ring formation of a number of natural β-lactones, such as Anisatin, Guaiagrazielolide, Papyriogenin, Hymeglusin, Vibralactone A, Omuralide, Vittatalactone, Spongiolactone, Ebelactone A, Obafluorin, Lipstatin, and Salinosporamide A. However, because β-lactone synthetases generally favor substrates with long alkyl tails, the resultant β-lactones exhibit the same and are generally bulky, highly aromatic, and often unsuitable candidates for the synthesis of PHAs. Rather, small molecule, short alkyl tail β-lactones are preferred for PHA synthesis.

### SUMMARY

In one or more aspects, the present disclosure provides a method including reacting a feedstock and a recombinant microorganism harboring one or more genes encoding enzymes involved in a metabolic pathway of a 3-hydroxycarboxylic acid synthesis, thereby producing a 3-hydroxycarboxylic acid having C₁-C₁₀ alkyl groups; and reacting the 3-hydroxycarboxilic acid with a bioengineered natural β-lactone synthetase, the bioengineered β-lactone synthetase engineered to comprise a substrate binding pocket capable of accepting the 3-hydroxycarboxylic acid having C₁-C₁₀ alkyl groups, thereby producing a β-lactone having C₁-C₁₀ alkyl groups.

### BRIEF DESCRIPTION OF THE DRAWINGS

To assist those of ordinary skill in the relevant art in making and using the subject matter hereof, reference is made to the appended drawings. The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 is a process flow diagram illustrating the two-step process of the present disclosure.

FIG. 2 illustrates non-limiting 3-hydroxycarboxylic acids and short alkyl tail β-lactones synthesized according to one or more aspects of the present disclosure.

### DETAILED DESCRIPTION

This application relates to beta-lactone production and, more particularly, to integrated bioconversion of beta-lactone production and methods related thereto.

As described above, naturally occurring β-lactones typically exhibit long alkyl tails that are not generally conducive to the synthesis of biodegradable PHAs. The present disclosure provides a biological mechanism for producing β-lactones characterized by short alkyl tails that have an affinity for the synthesis of PHAs. As described herein, biocatalysis using bioengineered natural β-lactone synthetase provides for the de novo biosynthesis of short alkyl tail β-lactone synthons for engineering PHAs with controllable and tunable properties.

More particularly, the present disclosure utilizes a two-step process in which small alkyl-group 3-hydroxycarboxylic acids are formed using genetically engineered microorganisms, followed by contact with a bioengineered natural β-lactone synthetase designed to have a binding pocket capable of accepting the aforementioned small alkyl-group 3-hydroxycarboxylic acids (the 3-hydroxycarboxylic acids described herein are recombinantly-produced, as described below), thereby forming short alkyl tail β-lactones conducive to PHA synthesis.

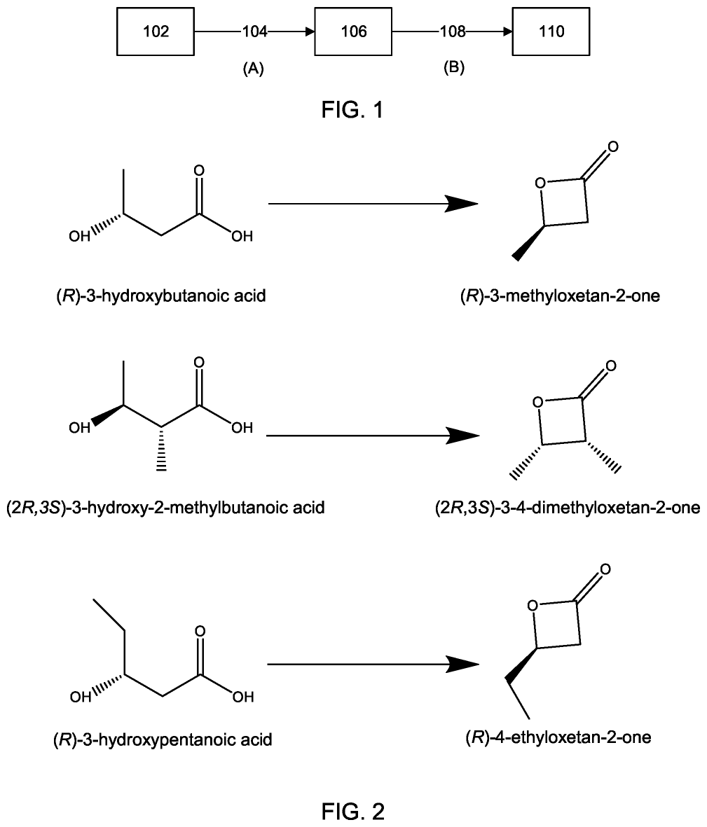

Referring now to FIG. 1, a flow diagram illustrating the two-step process of the present disclosure is provided. With reference to FIG. 1, the first step is illustrated as “(A)” and the second is illustrated as “(B).” In step (A), one or more feedstocks 102 are contacted with one or more bioengineered microorganism 104 to produce one or more 3-hydroxycarboxylic acids 106. That is, the bioengineered microorganism 104 is genetically modified to produce one or more desired (specific) 3-hydroxycarboxylic acids 106. The one or more 3-hydroxycarboxylic acids 106 are characterized as having small alkyl groups (e.g., C₁-C₁₀ alkyl groups). In step (B), the 3-hydroxycarboxylic acids 106 are contacted with one or more bioengineered β-lactone synthetase enzymes 108. The bioengineered β-lactone synthetase enzymes 108 are characterized as having a binding pocket for receiving the 3-hydroxycarboxylic acids 106. It is to be appreciated that the one or more 3-hydroxycarboxylic acids 106 of the present disclosure are not a natural substrate for natural β-lactone synthetase enzymes. An enzymatic reaction between the one or more recombinantly-produced 3-hydroxycarboxylic acids 106 and the one or more bioengineered β-lactone synthetase enzymes 108 results in the biocatalysis of short alkyl tail β-lactones 110. The biocatalysis described herein may be in vivo or ex vivo, as provided herein below.

In one or more aspects of the present disclosure, and with continued reference to FIG. 1, step (A) and step (B) may be performed in an integrated manner, such that they are performed back-to-back without significant pause (or any pause) between each step (e.g., without a storage step). Integration may save costs and time associated with any significant pause between each step. However, it is to be understood that, alternatively, each of step (A) and step (B) may be performed separately with a pause between each step (e.g., storage of the 3-hydroxycarboxylic acids prior to biosynthesis of the short alkyl tail β-lactones), without departing from the scope of the present disclosure.

In one or more aspects, the bioconversion shown in step (A) and step (B) of FIG. 1 may be based on the same microorganism (termed in vivo in the present disclosure) with the co-expression of multiple enzymes (e.g., enzymes for the metabolic pathway of producing 3-hydroxycarboxylic acid as described herein below and engineered to convert 3-hydroxycarboxylic acids to β-lactones). Alternatively or in addition, the bioconversion shown in step (A) and step (B) of FIG. 1 may be based on two different microorganism (e.g., two or more co-cultured engineered microorganisms wherein one or more synthesize 3-hydroxycarboxylic acids and one or more separate enzymes synthesize β-lactones) (termed ex vivo in the present disclosure). In yet one or more aspects of the present disclosure, alternatively or in addition, the bioconversion shown in step (A) and step (B) of FIG. 1 may be effected using a combination of bioengineered microorganism and an independent enzymatic bioconversion, metal organic framework (MOF)-based separation, absorption, organic solvent extraction, and the like, and any combination thereof.

Further, in one or more aspects, the produced short alkyl tail β-lactones according to the methodologies of the present disclosure may be further purified prior to synthesizing biodegradable PHA. Purification may include, but is not limited to, distillation, ion exchange, membrane separation.

In one or more aspects, the 3-hydroxycarboxylic acids of the present disclosure may have the following Formula I below:

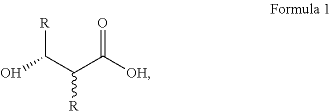

where R is an —H, —CₙH₂ₙ₊₁, or a hydrocarbon chain containing an aromatic group; and n is an integer in the range of from 1 to 10.

In one or more aspects, the short alkyl tail β-lactones of the present disclosure may have the following Formula 2 below:

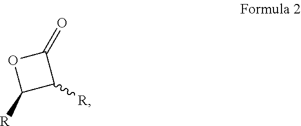

where R is an —H, —CₙH₂ₙ₊₁, or a hydrocarbon chain containing an aromatic group; and n is an integer in the range of from 1 to 10.

Referring now to FIG. 2, illustrated are combinations of 3-hydroxycarboxylic acids and resultant short alkyl tail β-lactones formed according to the methodology of the present disclosure. That is, the 3-hydroxycarboxylic acids are obtained using one or more feedstocks (102 of FIG. 1) in combination with one or more bioengineered microorganism (104 of FIG. 1).

Thereafter, the 3-hydroxycarboxylic acids are reacted with one or more bioengineered β-lactone synthetase enzymes 108 of FIG. 1).

With continued reference to FIG. 2, the left side of the arrows represents the 3-hydroxycarboxylic acids and the right side of the arrows represents the short alkyl tail β-lactones. Accordingly, (R)-3-hydroxybutanoic acid may be used according to the methodologies described herein to synthesize (R)-3-methyloxetan-2-one; (2R,3S)-3-hydroxy-2-methylbutanoic acid may be used according to the methodologies described herein to synthesize (2R,3S)-3-4-dimethyloxetan-2-one; and (R)-3-hydroxypentanoic acid may be used according to the methodologies of the present disclosure to synthesize (R)-4-ethyloxetan-2-one. It is to be appreciated that the examples provides in FIG. 2 are nonlimiting and other 3-hydroxycarboxylic acids may be synthesized according to the methodologies of the present disclosure for forming short alkyl tail β-lactones as described herein (per FIG. 1).

The illustrative short alkyl tail β-lactones shown in FIG. 2 are characterized by high ring-strain, without the long alkyl tails characterized by the aforementioned natural β-lactones (e.g., Anisatin, Guaiagrazielolide, and the like). As such, they are highly-reactive electrophilic scaffolds for the synthesis of biodegradable PHAs compared to natural β-lactones because the ring-opening polymerization biocatalysis necessary to form the PHAs is directly accessible (i.e., the long alkyl tails of traditional natural β-lactones are of no hindrance). Additionally, the properties of the PHA are directly impacted by the substitution pattern of the lactone. Lactones with long alkyl tails are expected to make PHAs with reduced crystallinity compared to PHAs with shorter alkyl tails. That is, the bioengineered β-lactone synthetase enzymes of the present disclosure are particularly advantageous because, as described above, natural and currently available β-lactone synthetase enzymes produce only long alkyl tail β-lactone, and fail to produce any short alkyl tail β-lactone having the advantageous qualities for synthesizing biodegradable PHAs. The bioengineered β-lactone synthetase enzymes of the present disclosure have heretofore been unknown.

The feedstock (step (A) of FIG. 1) of the present disclosure may include, but is not limited to, sugar, vegetable oil, crude oil, methane, syngas, lignocellulosic biomass, algae biomass, municipal solid waste, and the like, and any combination thereof.

The microorganism (step (A) of FIG. 1) of the present disclosure may include, but is not limited to, a prokaryotic organism (e.g., E. coli, B. subtilis, Corynebacterium sp., Pseudomonas sp., and the like), a eukaryotic organism (e.g., yeast, filamentous fungi, and the like), and any combination thereof.

The 3-hydroxycarboxylic acids (step (A) of FIG. 1) of the present disclosure may be, for example, de novo synthesized from a feedstock (e.g., one described herein), through depolymerization of biologically-produced PHA (in vivo or in vitro), through chemical synthesis. For example, genes encoding enzymes involved in the metabolic pathway of representative 3-hydroxycarboxylic acid, 3-hydroxybutyrate, include 3-ketothiolase (PhaA), β-ketoacyl-CoA thiolase, NADPH-dependent acetoacetyl-CoA reductase (PhaB), acetoacetyl-CoA reductase, PHA synthase (PhaC), PHA polymerase, poly(3-hydroxyalkanoate) depolymerase (Pha), PHA depolymerase, acyl-CoA thioesterase II (TesB), acyl-CoA thioesterase II, phosphotransbutyrylase-butyrate kinase (Ptb-Buk), and the like, and any combination thereof. The recombinant microorganisms of the present disclosure may be engineered to harbor any one or more of these enzymes.

The enzymatic conversion of 3-hydroxycarboxylic acid to form the short alkyl tail β-lactones (step (B) of FIG. 1) of the present disclosure is performed through biocatalysis using a bioengineered β-lactone synthetase enzymes. The energy source for fueling the biocatalysis may include, but is not limited to, adenosine triphosphate (ATP), adenosine diphosphate (ADP), pyrophosphate (PPi), thioester bonds, and the like, and any combination thereof. In one or more aspects of the present disclosure, the more efficient bioengineered β-lactone synthetase enzymes may be modified through, for example, rational design, random mutagenesis, and the like, and any combination thereof to have an engineered substrate binding pocket capable of accepting small 3-hydroxycarboxylic acid(s) with small alkyl groups (e.g., C₁-C₁₀ alkyl groups, such as shown in FIG. 2 and Formula 1).

In one or more aspects, the short alkyl tail β-lactones of the present disclosure may

be used to synthesize PHA by means of ring-opening polymerization. Ring-opening polymerization is a reaction in which a polymer chain comprises a reactive center on its terminal end that reacts with another cyclic monomer, thereby forming a longer polymer chain. In one or more aspects herein, the reactive center of the short alkyl tail β-lactones described herein may be anionic, cationic, or radical. The ring opening polymerization may be either catalyzed or uncatalyzed. In the case of catalyzed ring opening, the catalyst may be inorganic, organometallic, organic, enzymatic, or any combination thereof. In the case of catalyzed ring opening, the reactive center of the short alkyl tail β-lactones described herein is the bond between the growing polymer chain and the catalyst. Such a complex between the catalyst and the growing polymer chain may be anionic, cationic, or neutral, and may or may not be radical in nature.

### EXAMPLE EMBODIMENT

A nonlimiting example embodiment of the present disclosure include:

Embodiment A: A method comprising: reacting a feedstock and a recombinant microorganism harboring one or more genes encoding enzymes involved in a metabolic pathway of a 3-hydroxycarboxylic acid synthesis, thereby producing a 3-hydroxycarboxylic acid having C₁-C₁₀ alkyl groups; and reacting the 3-hydroxycarboxilic acid with a bioengineered natural β-lactone synthetase, the bioengineered β-lactone synthetase engineered to comprise a substrate binding pocket capable of accepting the 3-hydroxycarboxylic acid having C₁-C₁₀ alkyl groups, thereby producing a β-lactone having C₁-C₁₀ alkyl groups.

Nonlimiting example Embodiment A may include one or more of the following elements:

Element 1: wherein the feedstock is selected from the group consisting of sugar, vegetable oil, crude oil, methane, syngas, lignocellulosic biomass, algae biomass, municipal solid waste, and the like, and any combination thereof.

Element 2: wherein the microorganism is selected from the group consisting of a prokaryotic organism, a eukaryotic organism, and any combination thereof.

Element 3: wherein the microorganism is a prokaryotic organism selected from the group consisting of E. coli, B. subtilis, Corynebacterium sp., Pseudomonas sp., and any combination thereof.

Element 4: wherein the microorganism is a eukaryotic organism selected from the group consisting of yeast, filamentous fungi, and any combination thereof.

Element 5: wherein the enzymes are selected from the group consisting of 3-ketothiolase (PhaA), β-ketoacyl-CoA thiolase, NADPH-dependent acetoacetyl-CoA reductase (PhaB), acetoacetyl-CoA reductase, PHA synthase (PhaC), PHA polymerase, poly(3-hydroxyalkanoate) depolymerase (Pha), PHA depolymerase, acyl-CoA thioesterase II (TesB), acyl-CoA thioesterase II, phosphotransbutyrylase-butyrate kinase (Ptb-Buk), and any combination thereof.

Element 6: wherein the bioengineered β-lactone synthetase is engineered based on rational design or random mutagenesis.

Element 7: wherein the reacting of the 3-hydroxycarboxilic acid with a bioengineered natural β-lactone synthetase to produce the β-lactone is fueled by an energy source selected from the group consisting of adenosine triphosphate (ATP), adenosine diphosphate (ADP), pyrophosphate (PPi), thioester bonds, and any combination thereof.

Element 8: wherein the 3-hydroxycarboxylic acid comprises the formula:

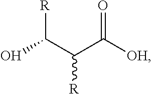

where R is an —H, —CₙH₂ₙ₊₁.

Element 9: wherein the produced β-lactone comprises the formula:

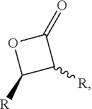

where R is an —H, —CₙH₂ₙ₊₁.

Element 10: wherein the 3-hydroxycarboxylic acid is (R)-3-hydroxybutanoic acid and the produced β-lactone is (R)-3-methyloxetan-2-one.

Element 11: wherein the 3-hydroxycarboxylic acid is (R)-3-hydroxybutanoic acid and the produced β-lactone is (R)-3-methyloxetan-2-one.

Element 12: wherein the 3-hydroxycarboxylic acid is (2R,3S)-3-hydroxy-2-methylbutanoic acid and the produced β-lactone is (2R,3S)-3-4-dimethyloxetan-2-one.

Element 13: wherein the 3-hydroxycarboxylic acid is (R)-3-hydroxypentanoic acid and the produced β-lactone is (R)-4-ethyloxetan-2-one.

Element 14: further comprising: synthesizing a polyhydroxyalkanoate (PHA) through ring-opening biocatalysis of the produced β-lactone.

Embodiment A may include any one, more, or all of Elements 1-14 in any combination.

Therefore, the disclosed methods and bioengineered β-lactone synthetase enzymes of the present disclosure are well adapted to attain the ends and advantages mentioned as well as those that are inherent therein.

As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The invention claimed is:

1. A method comprising:
reacting a feedstock and a recombinant microorganism harboring one or more genes encoding enzymes involved in a metabolic pathway of a 3-hydroxycarboxylic acid synthesis, thereby producing a 3-hydroxycarboxylic acid having C₁-C₁₀ alkyl groups; and
reacting the 3-hydroxycarboxilic acid with a bioengineered natural β-lactone synthetase, the bioengineered β-lactone synthetase engineered to comprise a substrate binding pocket capable of accepting the 3-hydroxycarboxylic acid having C₁-C₁₀ alkyl groups, thereby producing a β-lactone having C₁-C₁₀ alkyl groups.

2. The method of claim 1, further comprising synthesizing a polyhydroxyalkanoate (PHA) through ring-opening biocatalysis of the produced β-lactone.

3. The method of claim 1, wherein the feedstock is selected from the group consisting of sugar, vegetable oil, crude oil, methane, syngas, lignocellulosic biomass, algae biomass, municipal solid waste, and the like, and any combination thereof.

4. The method of claim 1, wherein the microorganism is selected from the group consisting of a prokaryotic organism, a eukaryotic organism, and any combination thereof.

5. The method of claim 1, wherein the microorganism is a prokaryotic organism selected from the group consisting of E. coli, B. subtilis, Corynebacterium sp., Pseudomonas sp., and any combination thereof.

6. The method of claim 1, wherein the microorganism is a eukaryotic organism selected from the group consisting of yeast, filamentous fungi, and any combination thereof.

7. The method of claim 1, wherein the enzymes are selected from the group consisting of 3-ketothiolase (PhaA), β-ketoacyl-CoA thiolase, NADPH-dependent acetoacetyl-CoA reductase (PhaB), acetoacetyl-CoA reductase, PHA synthase (PhaC), PHA polymerase, poly(3-hydroxyalkanoate) depolymerase (Pha), PHA depolymerase, acyl-CoA thioesterase II (TesB), acyl-CoA thioesterase II, phosphotransbutyrylase-butyrate kinase (Ptb-Buk), and any combination thereof.

8. The method of claim 1, wherein the bioengineered β-lactone synthetase is engineered based on rational design or random mutagenesis.

9. The method of claim 1, wherein the reacting of the 3-hydroxycarboxilic acid with a bioengineered natural β-lactone synthetase to produce the β-lactone is fueled by an energy source selected from the group consisting of adenosine triphosphate (ATP), adenosine diphosphate (ADP), pyrophosphate (PPi), thioester bonds, and any combination thereof.

10. The method of claim 1, wherein the 3-hydroxycarboxylic acid comprises the formula: 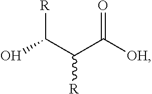
where R is an —H, —CₙH₂ₙ₊₁.

11. The method of claim 1, wherein the produced β-lactone comprises the formula: 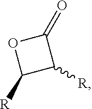
where R is an —H, —CₙH₂ₙ₊₁.

12. The method of claim 1, wherein the 3-hydroxycarboxylic acid is (R)-3-hydroxybutanoic acid and the produced β-lactone is (R)-3-methyloxetan-2-one.

13. The method of claim 1, wherein the 3-hydroxycarboxylic acid is (2R,3S)-3-hydroxy-2-methylbutanoic acid and the produced β-lactone is (2R,3S)-3-4-dimethyloxetan-2-one.

14. The method of claim 1, wherein the 3-hydroxycarboxylic acid is (R)-3-hydroxypentanoic acid and the produced β-lactone is (R)-4-ethyloxetan-2-one.
